(19) United States
(12) Patent Application Publication
(10) Pub. No.: US 2026/0315697 A1
(43) Pub. Date: Oct. 15, 2026

(54) A medicament cassette

(71) Applicant: SHL Medical AG, Zug (CH)

(72) Inventors: Marcus Söderlund, Nacka Strand (SE); Daniel Carlsson, Nacka Strand (SE); Mattias Myrman, Nacka Strand (SE); Daniel Säll, Nacka Strand (SE); Plamen Balkandjiev, Nacka Strand (SE)

(21) Appl. No.: 19/481,939

(22) PCT Filed: May 3, 2024
(86) PCT No.: PCT/EP2024/062170

§ 371 Date: Nov. 6, 2025

(30) Foreign Application Priority Data
May 17, 2023 (EP) 23173890.7

(51) Int. Cl. A61M 5/24; A61M 5/20

(52) U.S. Cl. CPC A61M 5/2422; A61M 5/20; A61M 2005/2407; A61M 2005/244; A61M 2005/2488; A61M 2205/121

(57) ABSTRACT

The present disclosure generally relates to medicament delivery devices such as autoinjectors or infusers, and particularly concerns a medicament cassette configured to accommodate a medicament container.

### TECHNICAL FIELD

The present disclosure generally relates to medicament delivery devices such as autoinjectors or infusers, and particularly concerns a medicament cassette configured to accommodate a medicament container.

### BACKGROUND

A number of medical conditions require injections. These days, a number of different injection devices exist, including various types of pen injectors, autoinjectors and infusion devices for large volume injections. Although many of these devices have enabled major improvements in the management of a number of medical conditions, various limitations do still exist in the current technology. Not least amongst these are the difficulties faced by patients that require frequent injections and by patients that need to inject particularly viscous drugs. In considering these problems, the applicant has appreciated that various developments could be made to help improve the medicament delivery devices on the market today, for example concerning more effective means to administer larger injection volumes, which are set out in more detail below.

### SUMMARY

An object of the present disclosure is to provide a medicament cassette for a medicament delivery device which solves, or at least mitigates problems of the prior art.

According to a first aspect of the present disclosure, there is provided a medicament cassette configured to accommodate a medicament container, the medicament cassette is attachable to a medicament delivery device for delivery of the medicament to an injection site via an infusion set connected to the medicament cassette, the medicament cassette reaching between a distal end and a proximal end along a longitudinal axis, the cassette comprising: a cassette body adapted to receive the medicament container in a cylindrical inner space of the cassette main body, the cassette body having a through-hole at a proximal end through which an adapter of the infusion set is connected to the medicament container; a rear cap comprising a rear end plate at its distal end; at least one slider arm extending in the proximal direction relative to the rear end plate of the rear cap; wherein the at least one slider arm is arranged inside the cassette body; an actuator movable with respect to the cassette body, the actuator is mechanically connected with the slider arm such that a movement of the actuator cause a sliding distal motion of the slider arm with respect to the cassette body.

Embodiments of the present disclosure advantageously provides for a medicament cassette that accommodates a medicament container. The medicament cassette is attachable to a medicament delivery device by insertion in the receiving interface to lock the medicament cassette in an attached position in the receiving interface. By means of the separate medicament cassette, variable medicament volumes are possible using the same medicament delivery device. Thus, the volume of the medicament container can range from smaller volumes of about 2 ml to larger volumes of about 10 ml or more depending on the size of the medicament cassette.

According to one embodiment, the actuator that can cause a sliding motion of the slider arm is advantageously used for starting and pausing injection. Once the medicament cassette is attached to the medicament delivery device and the slider arm is moved distally, the distal protruding position of the slider arm triggers the electrically powered medicament delivery device to start an injection by pushing a switch activator of the electrically powered medicament delivery device. Moving back the slider arm in proximal direction pauses the injection.

Furthermore, according to one embodiment, the slider arm can be a part of the rear cap. In this embodiment, the actuator that can cause a sliding motion of the rear cap is advantageously used for starting and pausing injection. Once the medicament cassette is attached to the medicament delivery device and the rear cap is moved distally, the distal protruding position of the rear cap triggers the electrically powered medicament delivery device to start an injection by pushing a switch activator of the electrically powered medicament delivery device. Moving back the rear cap in proximal direction pauses the injection.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the components thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, “circumferentially” refer to a circumference or a circumferential direction 301 relative to an axis 102, typically a central axis extending in the direction of the longest extension of the device and/or component. Similarly, “radial” or “radially” refer to a direction 302 extending radially relative to the axis, and “rotation”, “rotational” and “rotationally” refer to rotation relative to the axis.

According to one embodiment, the cassette body comprises at least one slots on opposing sides of the cassette body, the slots having their main extension parallel with the longitudinal axis of the medicament cassette.

According to one embodiment, the rear cap comprises at least one radially outwards extending protrusions positioned in the slots of the cassette body.

According to one embodiment, at least one slider arm is an integral part of the rear cap.

According to one embodiment, the rear cap may comprise at least one flexible arm, the at least one flexible arm extends inclined radially outwards for initially preventing distal motion of the rear cap with respect to the cassette body. The flexible arm may have a fixed proximal end and a free distal end where the free distal end is movable with respect to the fixed proximal end. Advantageously, the free end of the flexible arm abuts against a proximally facing surface of the cassette body which prevents the rear cap from moving distally before engagement with the receiving interface of the medicament delivery device.

According to one embodiment, the flexible arms may flex radially inwards by interaction with a receiving interface of the medicament delivery device, whereby the rear cap is released from the cassette body and is movable in the distal direction with respect to the cassette body. Advantageously, during an engagement procedure for connecting the medicament cassette to the medicament delivery device, the flexible arms are moved by interaction with the receiving interface, thereby releasing the rear cap to be allowed distal motion. The rear cap is advantageously not allowed to move distally prior to connecting it to the medicament delivery device to make sure the medicament container stays in place in the cassette body.

According to one embodiment, the rear cap may comprise the actuator being a sliding button interface for a user to slide the rear cap in the distal direction. In other words, the sliding button interface may be part of the rear cap, as in being integrated with the rear cap. Alternatively, the actuator, e.g., a sliding button interface, is a part of the slider arm. In another embodiment, the sliding button interface may comprise of parallel ribs on a surface of the rear cap that the user may push on distally to slide the rear cap in the distal direction.

It should be noted that, in one embodiment where the slider arm is an independent component relative to the rear cap, the movements of the rear cap as mentioned above are performed by the slider arm.

According to one embodiment, the rear cap may comprise a proximally facing surface to abut against a distal surface of the medicament container to prevent the medicament container from escaping out from the distal end of the cassette body. The rear cap comprises a through-hole for allowing a plunger rod of the medicament delivery device to enter the cassette body to compress the medicant container. The through-hole may be smaller than the outer dimensions of the medicament container thereby preventing the medicament container from falling out from the cassette body.

According to one embodiment, the at least one slot may comprise a first portion and a second portion and a narrowing portion between the first portion and a second portion. The narrowing portion is narrower than the outer dimension of the at least one radially outwards extending protrusion of the rear cap.

According to one embodiment, the at least one radially outwards extending protrusion of the rear cap may be longitudinally lockable in each of the first portion and the second portion. The narrowing portion provides a snap-lock feature for releasably locking the at least one radially outwards extending protrusion longitudinally in the first portion and in the second portion. The snap-lock feature provides a tactile feeling for the user when moving the rear cap between the two positions, where one position is with the rear cap distally protruding.

According to one embodiment, the cassette body may comprise a through-hole to receive the actuator in the form of a rotatable button, the rotatable button comprising at least one radial protrusion that reach the at least one slider arm of the rear cap inside the cassette body. The button may be a separable component from the rear cap. The rotatable button is assembled in the cassette body and rotated for moving the rear cap distally.

According to one embodiment, the at least one slider arm may comprise a cam surface that the radial protrusion abuts against when the rotatable button is rotated, the interaction between the radial protrusion of the rotatable button and the cam surface cause the distal motion of the rear cap.

According to one embodiment, the cassette body may comprise two grooves on a sleeve of the cassette body surrounding the through-hole, and the rotating button comprises a at least one protrusion that is releasably engageable in each of the two grooves in two different rotational orientations of the rotating button with respect to the through-hole. The at least one protrusion and the corresponding grooves provide for a snap-lock feature that provides tactile feedback to the user when the rotatable button is rotated for actuation.

According to one embodiment, the two rotational orientations may be about 90 degrees apart.

According to one embodiment, the slot may be uniformly shaped. That is, when the rotatable button is used, the slot for the protrusion of the rear cap may be uniform, without a narrowing portion.

According to one embodiment, the cassette body may comprise radially inwards extending protrusions configured to prevent the medicament container from escaping out from the distal end of the cassette body.

According to one embodiment, the rear cap may comprise a rear end plate with a longitudinal through-hole for receiving a plunger rod from the medicament delivery device.

According to one embodiment, the medicament cassette may be attachable to a proximal end of the medicament delivery device by insertion of the medicament cassette in the proximal receiving interface of the medicament delivery device, and a subsequent axial rotation of the medicament cassette, whereby a distally facing surface of the cassette body engages with a proximally facing surface of the receiving interface to longitudinally lock the medicament cassette in an attached position in the receiving interface.

Generally, all terms used in the claims are to be interpreted according to their ordinary meaning in the technical field, unless explicitly defined otherwise herein. All references to “a/an/the member, apparatus, component, means, etc.” are to be interpreted openly as referring to at least one instance of the member, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 is a perspective view of an electrically powered medicament delivery device, a medicament cassette, and an infusion set, according to embodiments of the present disclosure;

FIG. 2 is an exploded view of a medicament cassette according to embodiments of the present disclosure;

FIG. 3A-B are a perspective views of a rear cap according to embodiments of the present disclosure;

FIG. 4A-B are a perspective views of a cassette body according to embodiments of the present disclosure;

FIG. 5 is a perspective views of a medicament cassette according to embodiments of the present disclosure;

FIG. 6 is a perspective view of a receiving interface according to embodiments of the present disclosure;

FIG. 7 is a perspective view of a rear cap according to embodiments of the present disclosure;

FIG. 8A-B are a perspective views of a cassette body according to embodiments of the present disclosure;

FIG. 9 is a perspective view of a rotatable button according to embodiments of the present disclosure;

FIG. 10 is a perspective view of a medicament cassette according to embodiments of the present disclosure;

FIG. 11 is a perspective view of a medicament cassette according to embodiments of the present disclosure;

FIG. 12 is a perspective view of the proximal end of a medicament cassette according to embodiments of the present disclosure;

FIGS. 13A-B are two perspective views of a medicament cassette attached to the receiving interface of a medicament delivery device according to embodiments of the present disclosure;

FIGS. 14A-B are two perspective views of a medicament cassette attached to the receiving interface of a medicament delivery device when the rear cap has moved distally according to embodiments of the present disclosure;

FIG. 15A is a cross-section of a medicament cassette according to embodiments of the present disclosure;

FIG. 15B is an exploded view of spring holders and a spring according to embodiments of the present disclosure;

FIG. 16A is a cross-section of a medicament cassette according to embodiments of the present disclosure;

FIG. 16B is an exploded view of a distal spring holder and a spring according to embodiments of the present disclosure;

FIG. 17A is a cross-section of a medicament cassette according to embodiments of the present disclosure;

FIG. 17B is a perspective view of a disc-spring according to embodiments of the present disclosure;

FIG. 18A is a cross-section of a medicament cassette according to embodiments of the present disclosure;

FIG. 18B is a perspective view of a polymer spring according to embodiments of the present disclosure;

FIG. 19 is a cross-section of a medicament cassette according to embodiments of the present disclosure; and

FIG. 20 is a cross-section of a medicament cassette according to embodiments of the present disclosure.

FIGS. 21-30 show perspective views of a medicament cassette according to another embodiments of the present disclosure.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

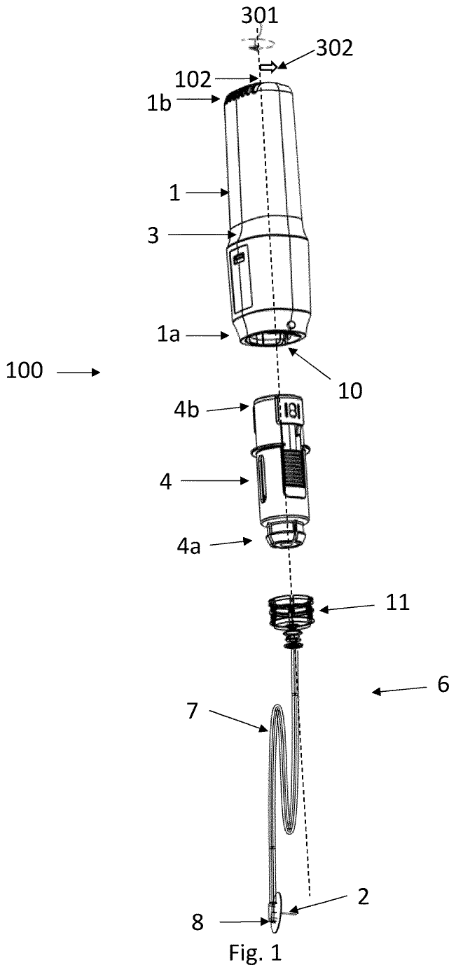

FIG. 1 shows an example of an electrically powered medicament delivery device 1 such as an autoinjector of an injection device, or an infusion device 100 according to embodiments of the present disclosure. The medicament delivery device 1 is configured to expel medicament from a medicament container via a medicament delivery member such as a needle 2, to a user at a dose delivery site. The medicament delivery device 1 extends from a proximal end 1a to a distal end 1b relative to the axis 102.

The medicament delivery device 1 comprises a housing 3 and is connectable to a medicament cassette 4 having a proximal end 4a and a distal end 4b.

The medicament cassette 4 is connectable to an infusion set 6 comprising a flexible tube 7 connected to a through-hole of the medicament cassette 4 at the proximal end 4a. At the other end of the tube 7 is the needle 2 attached via a pad 8.

The medicament cassette 4 is adapted to accommodate a medicament container which can be of different volumes, for example ranging from about 1 ml to about 10 ml.

The medicament delivery device 1 comprises a receiving interface 10 configured to received and lock the disposable cassette to the proximal end 1a of the medicament delivery device 1.

The infusion set 6 includes an adapter 11 for attaching the infusion set to the proximal end 4a of the medicament cassette 4 so that medicament from a medicament container housed in the medicament cassette 4 can be injected into the infusion set.

In one example, the flexible tube comprises a connector at its distal end. The connector of the tube is configured to be connected to the adapter 11. In a preferred example, the adapter 311 is configured to engaged with the connector of the tube via a thread 311c as shown in FIG. 23. In a preferred example, the adapter 311 comprises a protruding channel 311b configured to be at least partially positioned within the distal end of the tube.

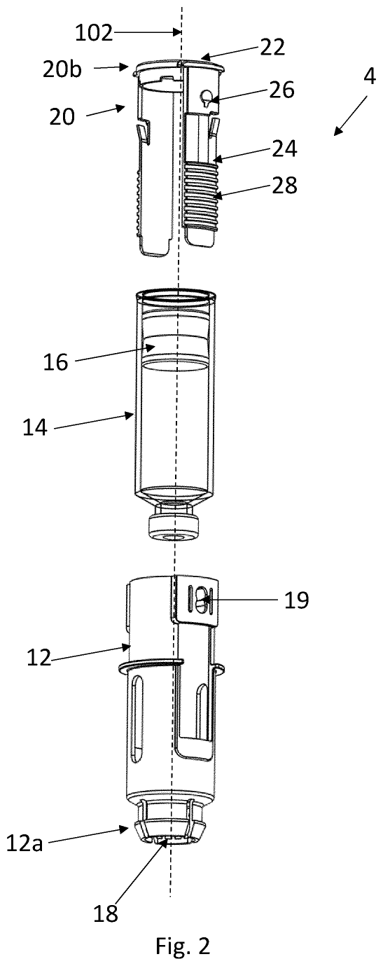

FIG. 2 is an exploded view of a medicament cassette 4 according to embodiments of the present disclosure.

The medicament cassette 4 comprises a cassette body 12 adapted to receive a medicament container 14 in a cylindrical inner space of the cassette body 12. A plunger rod adapter 16, such as a rubber stopper, may be fitted in the tubular medicament container 14. The plunger rod adapter 16 is adapted to be pushed proximally by a plunger rod of the medicament delivery device during a medicament delivery event. The proximal motion of the plunger rod adapter 16 cause expulsion of a medicament from the medicament container 14.

The cassette body 12 has a through-hole 18 at a proximal end 12a at which the adapter 11 of the infusion set 6 is connected to the medicament container 14. For example, the infusion set 6 is attachable to the medicament container 14, as disclosed in the examples below. Alternatively, the infusion set, namely, the adapter 11, the tube 7 are pre-assembled to the medicament container.

In one example, the cassette body 12 further comprises at least one slot 19 having a main extension parallel with the longitudinal axis 102 of the medicament cassette 4.

The medicament cassette 4 comprises a rear cap 20 comprising a rear end plate 22 at its distal end 20b and at least one slider arm 24 extending in the proximal direction from the distal end 20b of the rear cap 20. The rear cap 20 further comprises at least one radially outwards extending protrusions 26 that are arranged to be positioned in a respective slot 19 of the cassette body 12. When assembled, the at least one slider arm 24 of the rear cap 20 are arranged inside the cassette body 12 and interleaved between the medicament container 14 and the cassette body 12.

In should be note that the slider arm 24 can be an integral part of the rear cap 20; alternatively, the slider arm 24 can be an independent component of the rear cap, in this example, the rear cap can be immovably attached to the cassette body 12. Preferably, in one example where the slider arm can be an independent component of the rear cap, the slider arm is configured to move distally relative to the rear cap to interact with the medicament delivery device 1.

The medicament cassette 4 further comprises an actuator 28 movable with respect to the cassette body 12. The actuator 28 is mechanically connected with the rear cap 20 such that a movement of the actuator 28 causes a sliding distal motion of the rear cap 20 with respect to the cassette body 12.

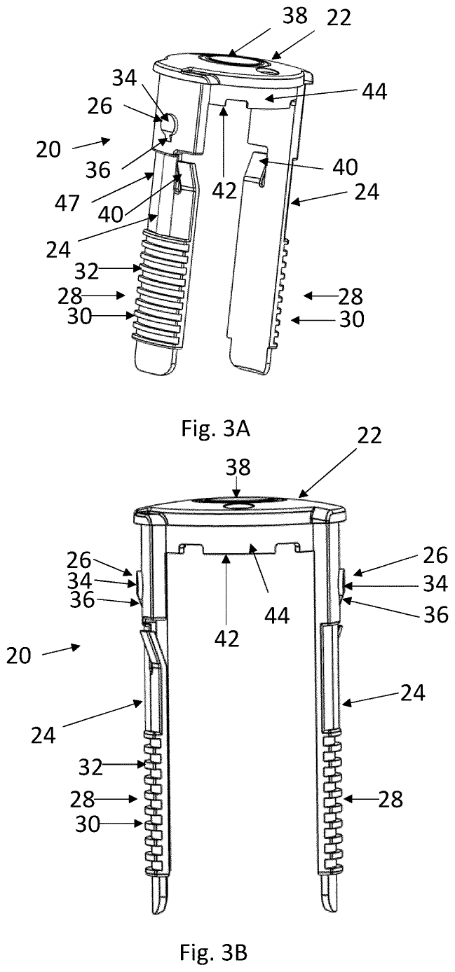

FIG. 3A-B are perspective views of a rear cap 20 according to embodiments of the disclosure. The rear cap 20 comprises two opposing slider arms 24. Each of the slider arms 24 comprises an actuator 28 being a sliding button interface 30 for a user to slide the rear cap 20 in the distal direction. The sliding button interface 30 comprises a set of transversal ribs 32 so that a user can get a better grip on the sliding button interface 30 with a finger.

The rear cap 20 further comprises two opposing radially outwards extending protrusions 26 that are intended to fit in a respective slot of the cassette body 12. The protrusions 26 comprise a rounded shape portion 34. On a proximal side of the protrusions 26 an inclined surface 36 that is inclined with respect to the longitudinal axis 102 with a slope that joins the radial outermost part of the protrusion 26. The inclined surface 36 facilitates fitting the protrusions 26 in the slots 19 of the cassette body when mounting of the rear cap 20 in the cassette body 12.

A longitudinal through-hole 38 in the rear end plate 22 allows for a plunger rod from the medicament delivery device to penetrate inside the medicament cassette to expel medicament from the medicament container 14.

Furthermore, the rear cap 20 comprising at least one flexible arm 40, preferably one flexible arm 40 for each slider arm 24. The at least one flexible arm 40 extends inclined radially outwards for initially preventing distal motion of the rear cap 20 with respect to the cassette body 12.

In some embodiments, the rear cap 20 includes a proximally facing surface 42 that is part of an annular flange 44 attached to the rear plate 22. The proximally facing surface may abut against a distal surface of the medicament container 14 to prevent the medicament container from escaping out from the distal end 20b of the cassette body. In other words, the diameter of the annular flange 44 is smaller than the diameter of the medicament container 14.

The rear cap 20 comprises at least one ridge 47 that is configured to releasable lock the medicament cassette 4 in a bayonet coupling of the receiving interface 10 of the medicament delivery device 1.

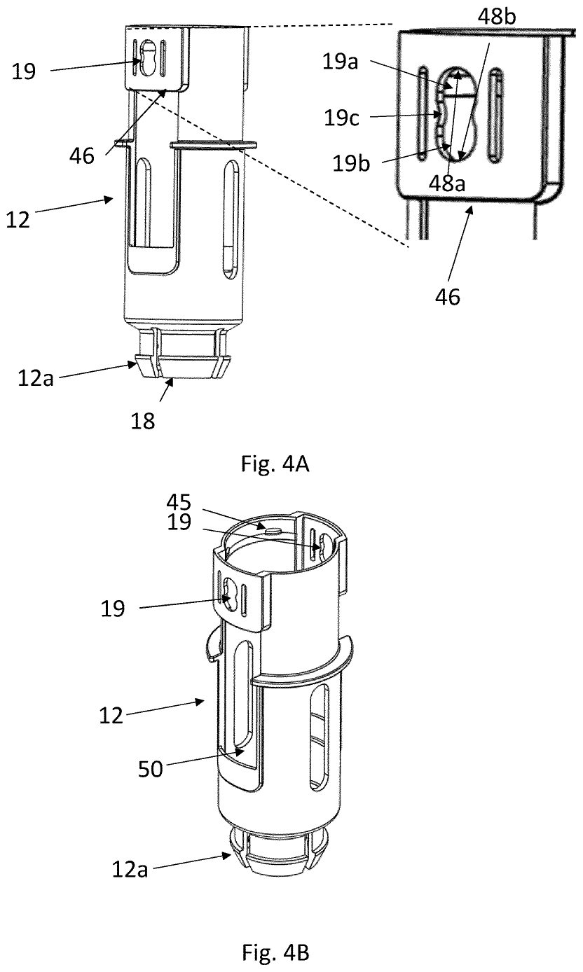

FIGS. 4a-b are perspective views of a cassette body 12 according to one embodiment.

The cassette body 12 comprises a proximally facing surface 46 on which the free end of the flexible arms 40 abut so that the rear cap 20 is prevented from moving distally with respect to the cassette body 12. This further ensures that the medicament container 14 is secured inside the medicament cassette 4.

The cassette body 12 further comprises opposing slots 19, each comprising a comprises a first portion 19a, a second portion 19b, and a narrowing portion 19c between the first portion and a second portion. The first portion 19a and the second portion 19b are round in shape to accommodate the round shaped protrusion 26 of the rear cap 20. Furthermore, the transversal width of the slot 19 at the narrowing portion 19c is smaller than the corresponding width at the first portion 19a and the second portion 19b. In addition, the width of the narrowing portion 19c is smaller than the width of the round shaped protrusion 26 of the rear cap 20, thereby providing a snap-in interface between the rear cap 20 including the sliding button interface 30 and the cassette body 12.

The snap-in interface between the rear cap 20 and the cassette body 12 provides for a longitudinal locking of the radially outwards extending protrusions 26 of the rear cap 20 in each of the first portion 19a and the second portion 19b. That is the protrusions 26 may abut against the proximally facing surface 48a of the first portion 19a or the distally facing surface 48b of the second portion 19b which limits the longitudinal motion between the rear cap 20 and the cassette body 12.

The cassette body 12 may optionally further comprise radially inwards extending protrusions 45 configured to prevent the medicament container 14 from escaping out from the distal end of the cassette body 12.

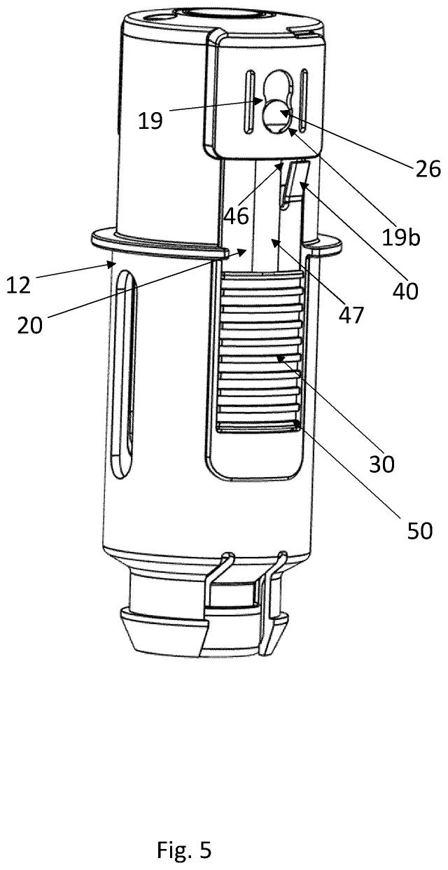

FIG. 5 is a perspective view of the medicament cassette 4. The rear cap 20 is mounted in the cassette body 12 and in this configuration the rear cap 20 is in its distal most arrangement with the protrusion 26 in the second portion 19b of the slot 19. Furthermore, the flexible arm 20 is in its radial outer position to abut against the proximally facing surface 46 of the cassette body 12 if the rear cap 20 is attempted to be moved distally. The sliding button interface 30 is accessible through the opening 50 in the cassette body 12.

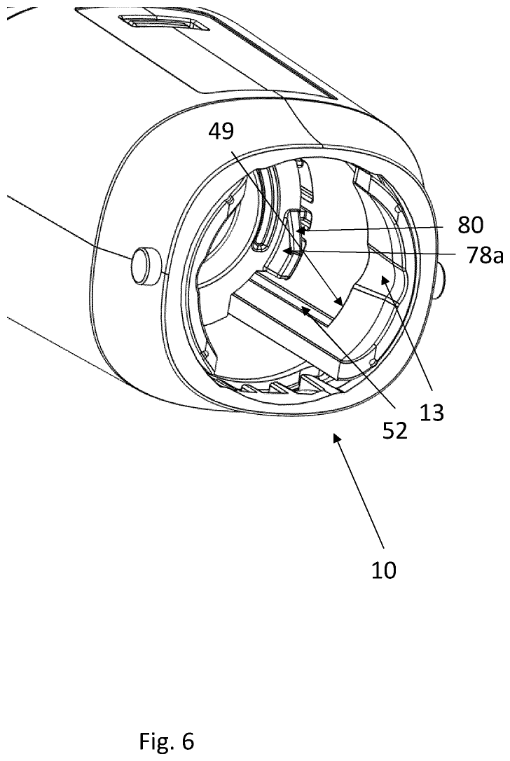

FIG. 6 is a perspective view of the receiving interface 10 of a medicament delivery device 1. The receiving interface 10 allows for a bayonet locking of the medicament cassette 4 in the interface 10. In one example, the medicament cassette 4 is inserted into the receiving interface 10 and is axially rotated clockwise in the perspective shown in FIG. 6 until the ridge 47 of the rear cap 20 reaches a groove 13 or trench of the receiving interface 10. 15. In this position the proximally facing surface 46 of the cassette body 12 engages with a proximally facing surface 49 of the receiving interface 10 to longitudinally lock the medicament cassette 4 in an attached position in the receiving interface 10.

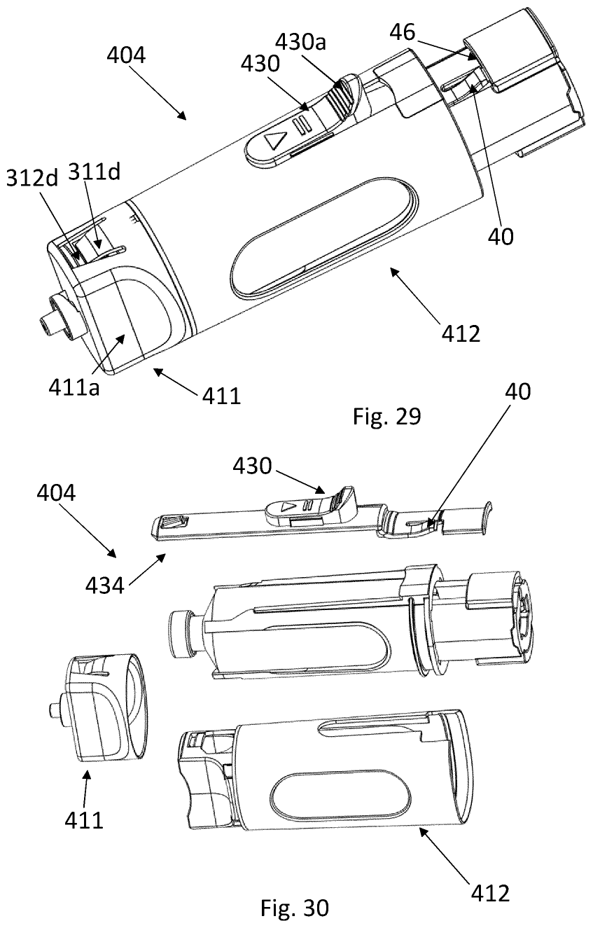

In this position, the radially outwards extending flexible arm 40 of the rear cap is bent or pushed inwards by the inclined surface 52 thereby the rear cap 20 is allowed to move in the distal direction by actuation using the slider button interface 30. In this way, the flexible arms 40 flex radially inwards by interaction with the receiving interface 10 of the medicament delivery device 1, whereby the rear cap 20 is movable in the distal direction with respect to the cassette body 12. As shown in FIG. 21 and FIG. 29, the lock formed by the proximally facing surface 46 of the cassette body 12 and the flexible arms 40 can be used in other embodiments that will be described below.

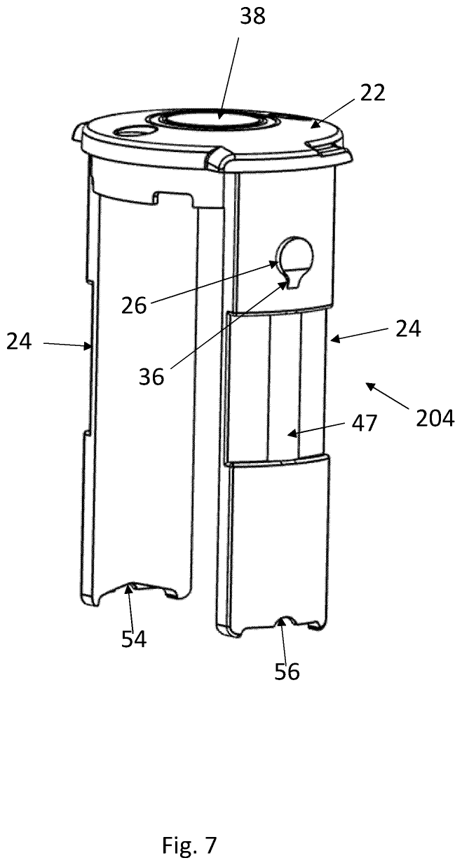

FIG. 7 is a perspective view of a rear cap 220 according to an embodiment. The rear cap 220 shares several features with the rear cap 20 that will not be repeated here. However, the rear cap 220 does not have a flexible arm 40 described above.

The at least one slider arm 24 of the rear cap 220 comprise a respective cam surface on a proximal end of the slider arm 24. Furthermore, the slider arms 24 comprise a locking groove 56 or cavity.

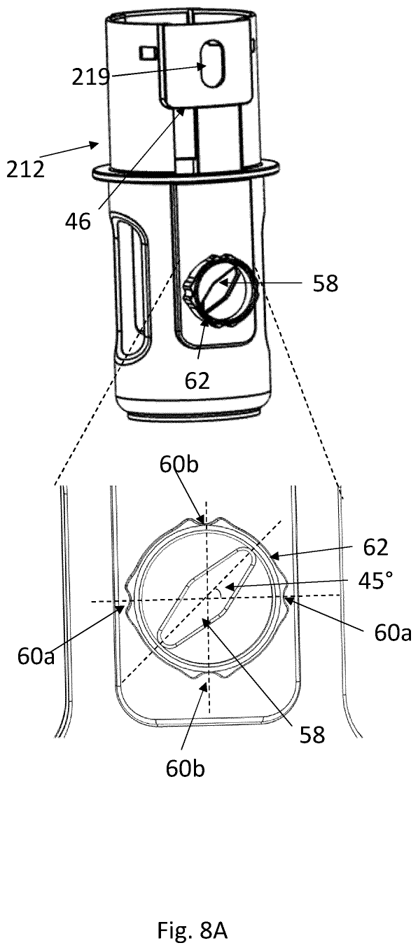

FIG. 8A is a perspective view of a cassette body 212. In this embodiment, the slot 219 is uniformly shaped and is configured to receive the protrusion 26 of the rear cap 220.

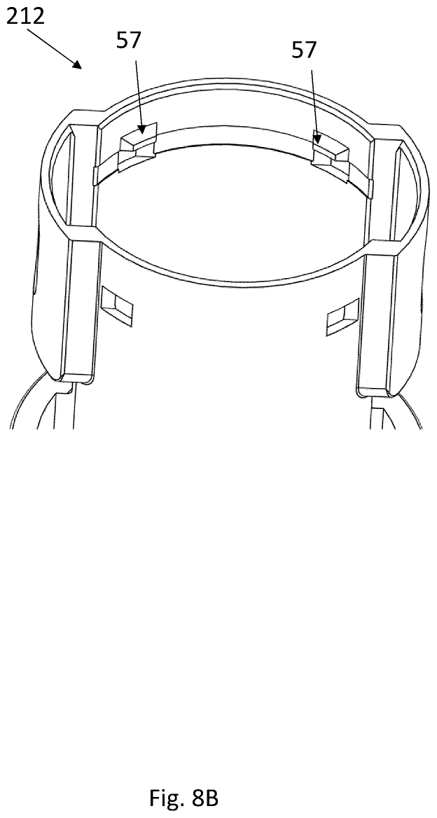

Further, the cassette body 212 comprises a through-hole 58 to receive the actuator in the form of a rotatable button described in FIG. 9. The cassette body 212 comprises two grooves 60a, 60b on a sleeve 62 of the cassette body 212. The sleeve 62 surrounds the through-hole 58 and allows for rotating the button in the sleeve 62. The two rotational orientations are about 90 degrees apart.

FIG. 8B is a perspective view of the distal end of the cassette body 212. The cassette body 212 comprises radially inwards extending protrusions 57 configured to prevent the medicament container from escaping out from the distal end of the cassette body. A proximally facing surface of the protrusions 57 may abut against the medicament container when it reaches the protrusions 57. The radially inwards extending protrusions 57 flex radially outwards to allow for the medicament container to be inserted into the cassette body 212.

FIG. 9 is a perspective view of a rotatable button 64. The rotatable button comprises a handle 66, an intermediate part 58, and a set of opposing radial protrusions 70a-b. The intermediate part 58 links the handle 66 with the protrusions 70a-b. The protrusions 70a-b form a shape that is similar to but fits through the through-hole 58 in the sleeve 62 of the cassette body 212. The intermediate part 58 is disc-shaped so that the rotatable handle 64 is conveniently rotated in the sleeve 62 when the protrusions 70a-b have been inserted through the through-hole 58.

When the rotatable button 64 is initially inserted in the through-hole 58 it is not locked in place. In this embodiment, a 45 degree clock-wise rotation results in that radially inwards extending protrusions 72a,b of the handle 66 are snap-fitted in the grooves 60a of the sleeve 62. This further results in an misalignment between the shape of the through-hole 58 and the protrusions 70a-b, so that the rotatable button 64 is locked in the though-hole 58 but may still be rotated.

If the rotatable button 64 is further rotated the protrusions 72a-b are releasable engageable in the grooves 60b. In other words, the protrusions 72 a-b are releasably engageable in the respective groove 60a, b in two different rotational orientations of the rotating button 64 with respect to the through-hole 58.

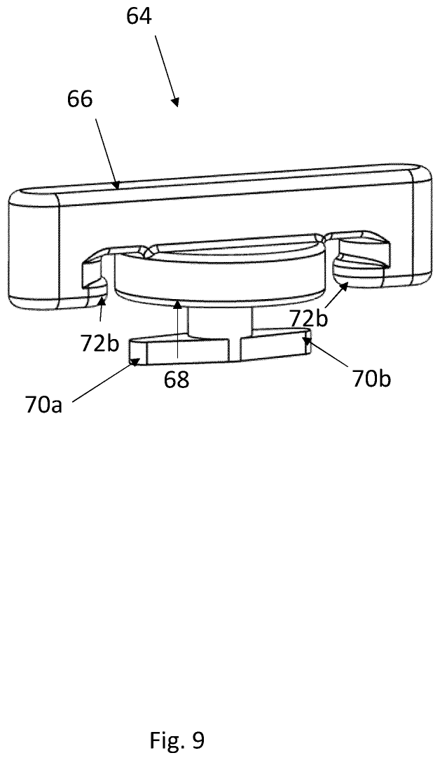

FIG. 10 is a perspective view of the medicament cassette 204 comprising the rear cap 220, the cassette body 212, and the rotatable button 64 rotated in the initial 45-degree position with the protrusions 72 a-b in the grooves 60a. The medicament cassette 204 locks in the receiving interface 10 of the medicament delivery device via the bayonet coupling.

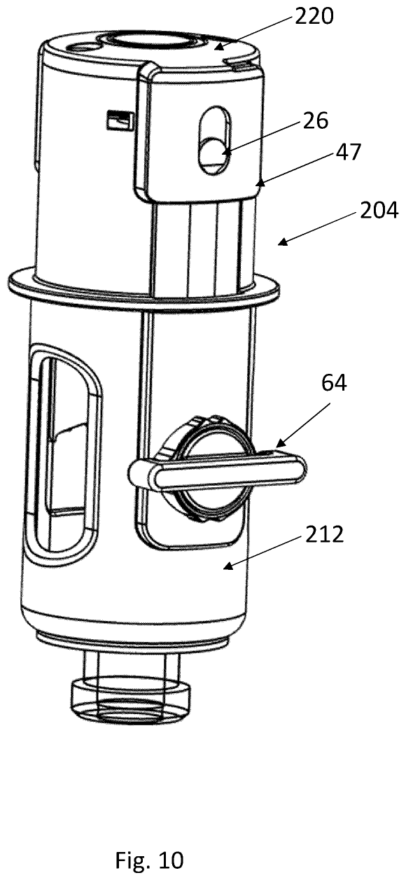

FIG. 11 illustrates a bottom view of the medicament cassette 204 with the medicament container removed. The radial protrusions 70a-b of the rotatable button 64 reach the at least one slider arm 24 of the rear cap 220 inside the cassette body 212. When the rotatable button 64 is rotated from the 45-degree position towards a 90-degree position where the protrusions 72a-b are engaged in the grooves 60b, one of the protrusions 70a-b abuts against the cam surface 54 which causes the distal motion of the rear cap 220.

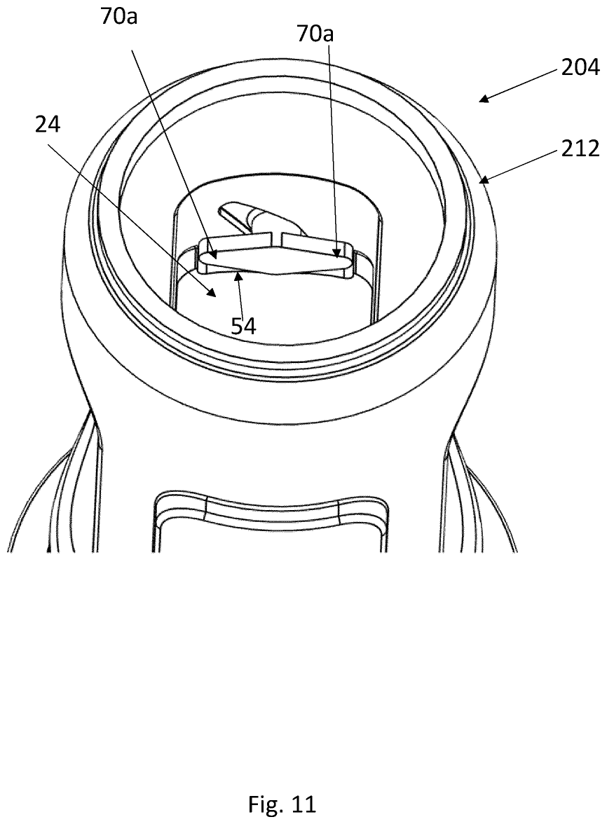

Turing to FIG. 12 which illustrates the proximal end 4b of a medicament cassette 4, 204. Regardless of whether the actuator is the rotatable button 64 of the slider button 30, the actuation causes the rear cap 20, 220 to move distally with respect to the cassette body 12, 212, as shown in FIG. 12 where the rear cap 20, 220 has moved a distance D with respect to the cassette body 12, 212.

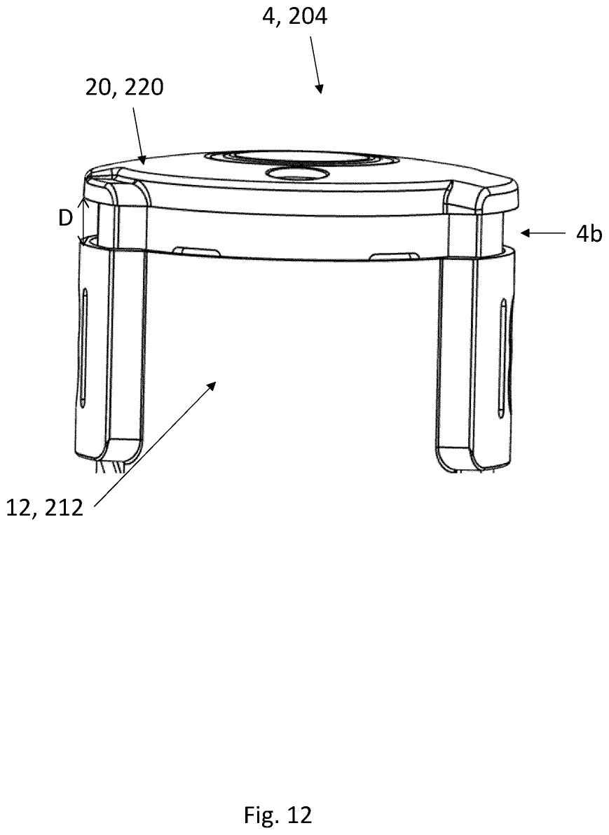

FIGS. 13A-B are two perspective views of a medicament cassette 4 attached to the receiving interface 10 and in contact with a switch activator 74 of the medicament delivery device 1. The switch activator 74 comprises two opposing arms 76a-b each with a respective proximal protrusion 78a-b comprising an inclined surface 80. One of the protrusions 78a and its inclined surface 80 are also shown in FIG. 6 inside the receiving interface 10. When the medicament cassette is rotated into a locking position in the receiving interface, the inclined surfaces 80 interact with the side surfaces 82 of a protrusion 84 of the rear cap 20 (or rear cap 220).

The interaction between the inclined surfaces 80 and the corresponding side surface 82 causes the switch activator 74 to move distally. The first 44 and second distally extending arms 76a-b each comprise a cut-out 86a-b on their respective distal ends 88a-b arranged diagonally with respect to each other so that the arms 76a-b are asymmetric. In FIGS. 13A-B the switch activator 74 to has moved distally so that the arm 76a has activated switch 90, whereas an opposing switch 92 is in the cut-out 88b and is thus not yet activated. Activation of the first switch 90 may for example cause activation of an electronic reader of the medicament delivery device 1 to read an electronic tag of a medicament container in the medicament cassette 4.

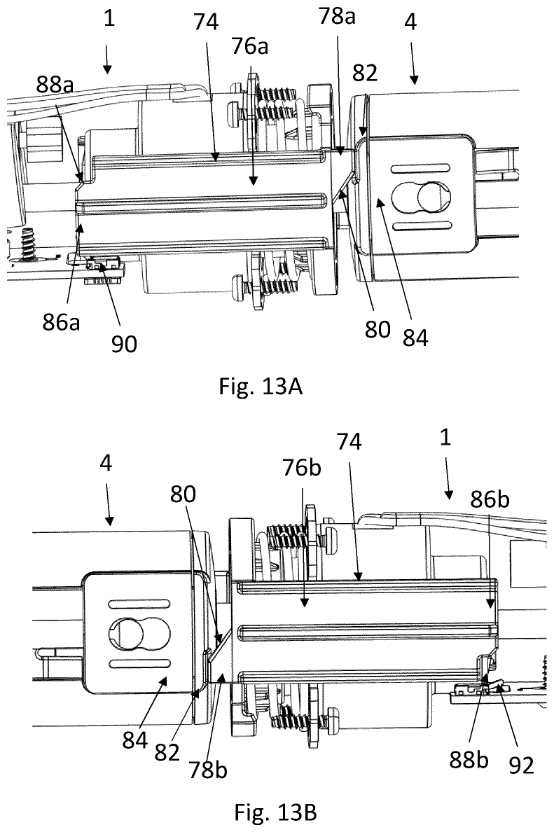

FIG. 14A-B are two perspective views of a medicament cassette 4 attached to the receiving interface 10 and when the actuator of the medicament cassette 4 has cause the distal motion, a distance D, of the rear cap 20. The protrusions 26 have move to the more distal portion of the slot 19. In this embodiment, the protrusions 26 have moved from the second portion 19b to the first portion 19a of the slot 19. The distal motion of the rear cap 20 push the switch activator further in the distal direction so that the distally extending arm 76b activate switch 92 of the medicament delivery device 1 which controls a plunger rod of the medicament delivery device 1 to apply a force on a medicament container in the medicament cassette 4 the cause expulsion of medicament for an injection. If the actuator 30, 64 of the medicament cassette 4, 204 is reversed so that the rear cap 20, 220 is moved in the proximal direction with respect to the cassette body 12, 212, the switch 92 is deactivated and the injection is paused.

The plunger rod of the medicament delivery device 1 may be driven by a stepper motor powered by a battery. The stepper motor is current-controlled and with a current locked to a high enough value for various type of injections. This means that the energy consumption is close to constant regardless of the motor speed and the mechanical work done by the motor. To provide long injection times, such as 10 minutes or even longer means that the motor will run at low speed resulting in very low efficiency in terms of mechanical work and/or electrical energy consumption and this may lead to overheating the motor. Furthermore, at very low motors speed detecting stall of the motor (Stallguard) is difficult. Running without stallguard risks losing a multitude of motor steps without being able to detect and compensate for them. This in turn could mean that the injection is not completed, as the software would falsely assume that the plunger rod has reached its end position, while in fact there are steps lost on its way.

To alleviate this problem, the medicament cassette according to anyone of the herein described embodiments may comprise a resilient member arranged in the medicament cassette along the longitudinal axis. The resilient member is compressible by the force provided by the plunger rod of the medicament delivery device during an injection, and is configured to decompress to allow the plunger rod to move proximally during motor pause time.

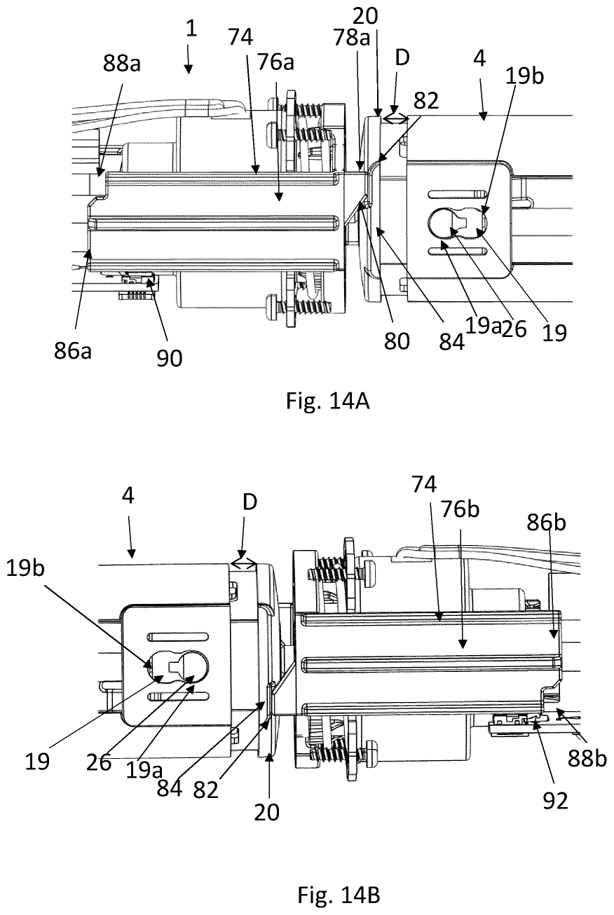

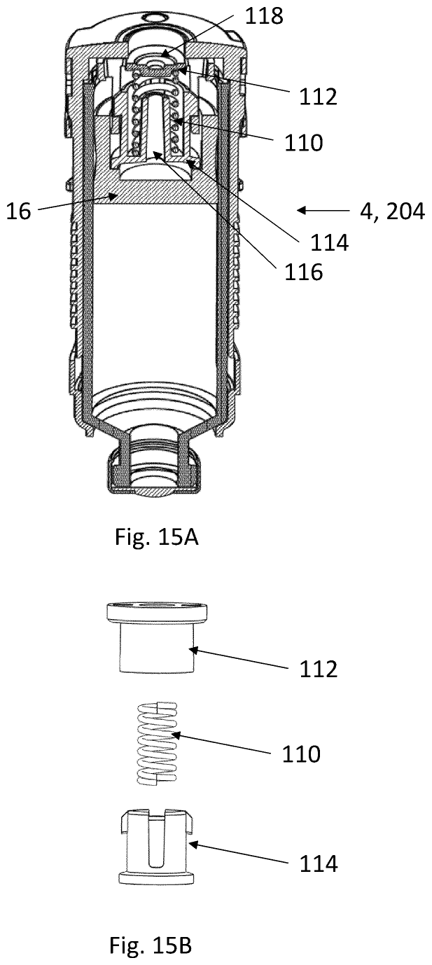

In one embodiment shown in the cross section of FIG. 15A, the resilient member is a spring 110 arranged, optionally pre-tensioned, between a distal spring holder 112 and a proximal spring holder 114. FIG. 15B is an exploded view of the spring holders 112, 114 and the spring 110. The proximal spring holder 114 comprises a central cylinder 116 on which the spring 110 is threaded. The plunger rod of the medicament delivery device 1 push on the distally facing surface 118 of the distal spring holder 112 to move the rubber stopper 16 proximally. Once the motor stops moving the plunger rod forward, the spring 110 decompress between the spring holders 112, 114 to move the rubber stopper further proximally thereby limiting the maximum force on the lead screw of the step motor. This also allows for an additional safety margin to prevent stalling the motor as well as increasing the efficiency of the system if the compression stroke of the spring is larger than the intended motor stroke.

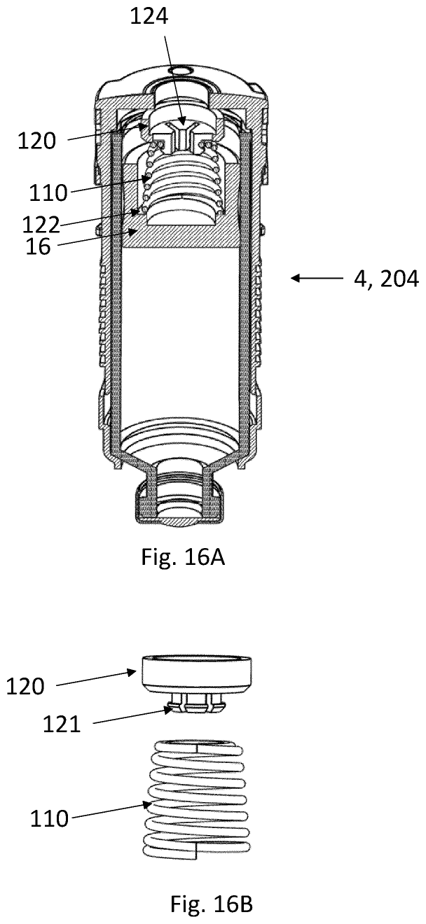

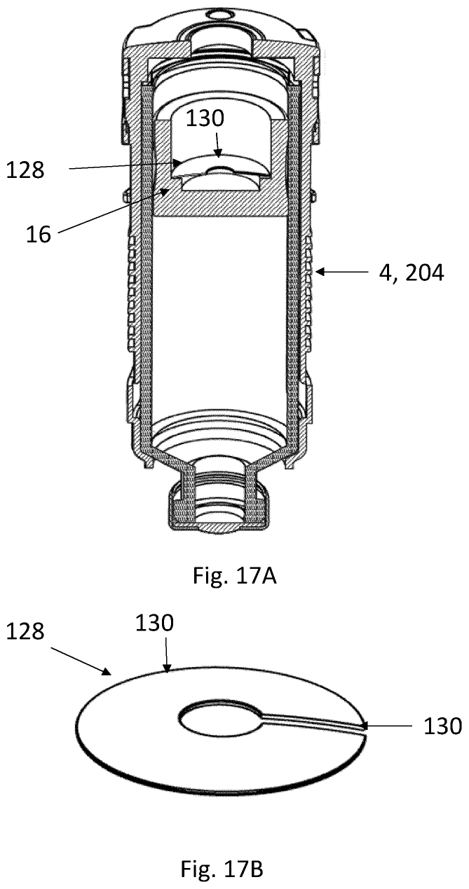

In one embodiment shown in the cross section of FIG. 16A, the resilient member is a spring 110 arranged, optionally pre-tensioned, between a distal spring holder 120 and directly abutting on a distally facing surface 122 of the rubber stopper 16. FIG. 16B is an exploded view of the somewhat conical spring 110 and the distal spring holder 120. The distal spring holder 120 comprises a proximal spring adapter 121 that is snap-fitted longitudinally into the spring. The plunger rod of the medicament delivery device 1 push on the distally facing surface 124 of the distal spring holder 120 to move the rubber stopper 16 proximally. Once the motor stops moving the plunger rod forward, the spring 110 decompress between the distal spring holder 120 and the rubber stopper 16 to move the rubber stopper 16 further proximally.

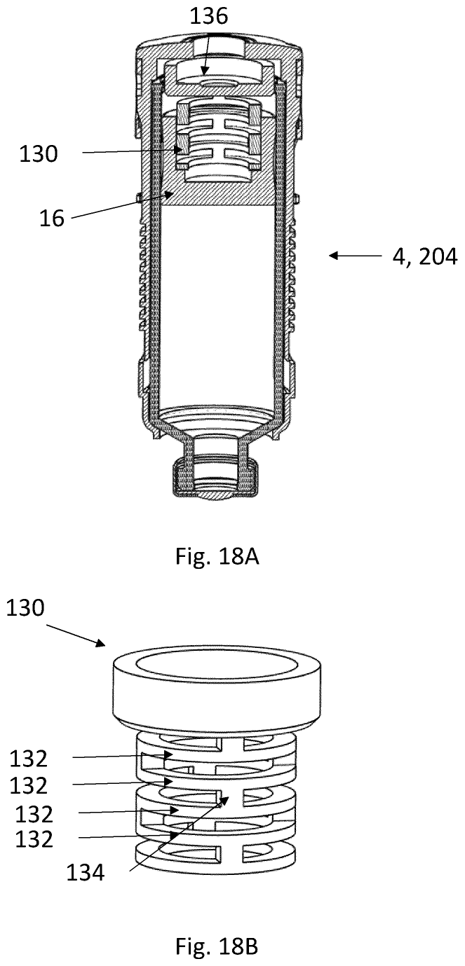

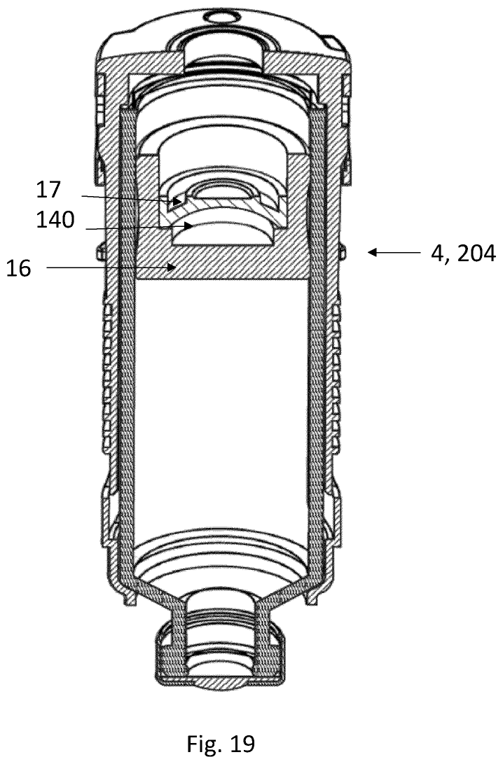

In one embodiment shown in the cross section of FIG. 17A, the resilient member is a disc spring 128 press-fitted into a cavity of the rubber stopper 16. The disc spring 128 is somewhat curved to provide the resilience of a spring. FIG. 17B is a perspective view of the disc spring 128 which comprises a radial cut 130 to facilitate the deformation of the metal disc-spring during compressions. In use, the plunger rod of the medicament delivery device 1 push on the distally facing surface 130 of the distal spring holder 112 to move the rubber stopper 16 proximally. Once the motor stops, the disc spring 128 decompress between the plunger rod and the rubber stopper 16 to move the rubber stopper 16 further proximally.

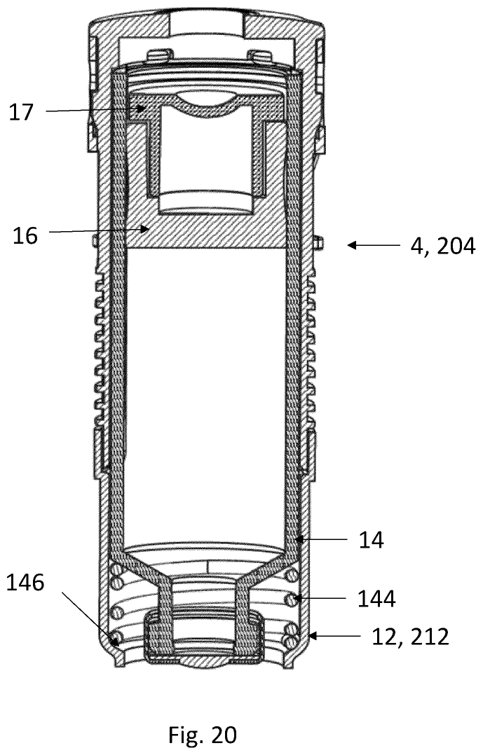

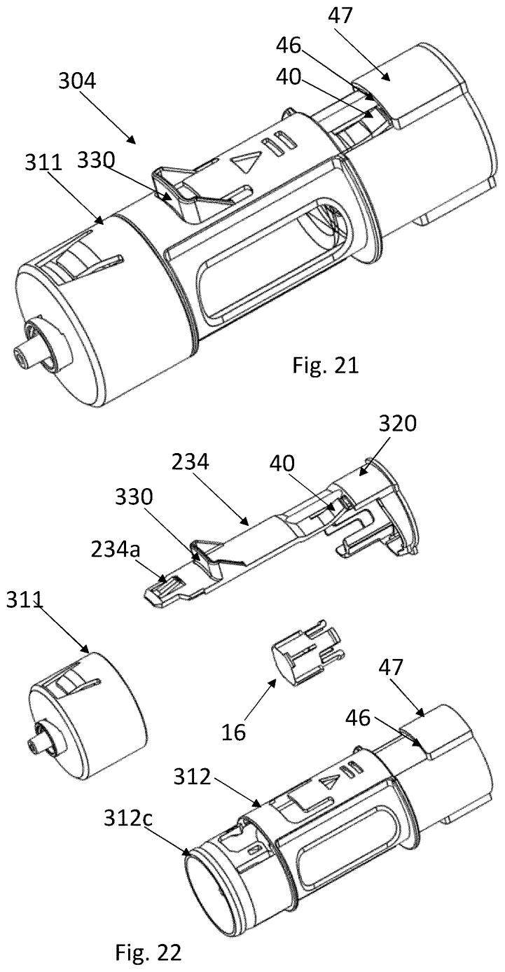

In one embodiment shown in the cross section of FIG. 18A, the resilient member is a polymer spring 130 fitted into a cavity of the rubber stopper 16. FIG. 18B is a perspective view of the polymer spring 130 which comprises a set of parallel rings 132 interconnected by ribs 134. In use, the plunger rod of the medicament delivery device 1 push on the distally facing surface 134 of the polymer spring 130 to move the rubber stopper 16 proximally. Once the motor stops, the polymer spring 130 decompress between the plunger rod and the rubber stopper 16 to move the rubber stopper 16 further proximally.

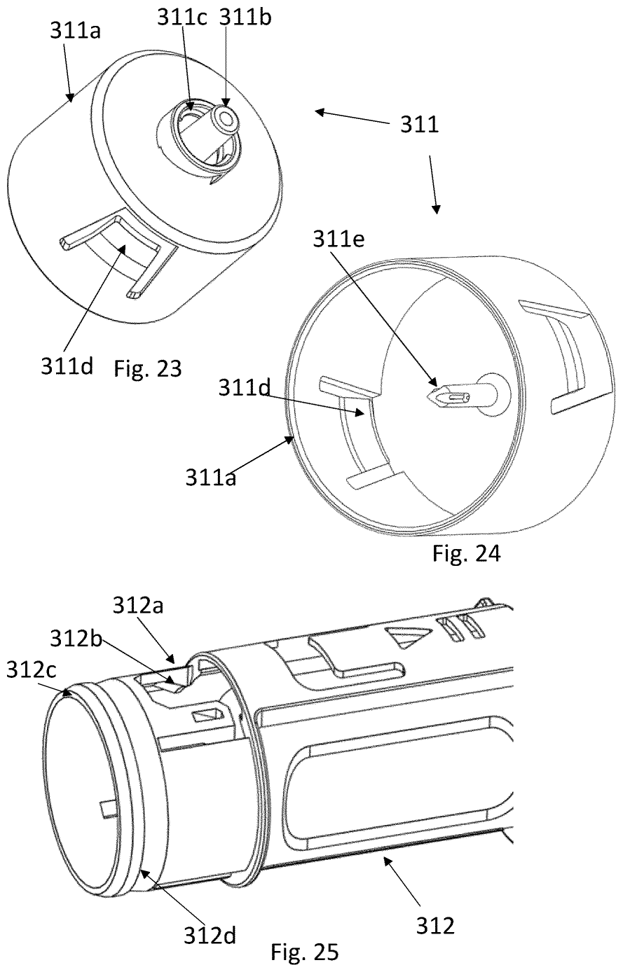

In one embodiment shown in the cross section of FIG. 19, the resilient member is a deformation cavity 140 of the rubber stopper 16. The deformation cavity is formed between the plunger rod 142 and the rubber stopper 16. In this way, part of the rubber stopper 16 is allowed to flex as the plunger adapter 17 press on it.

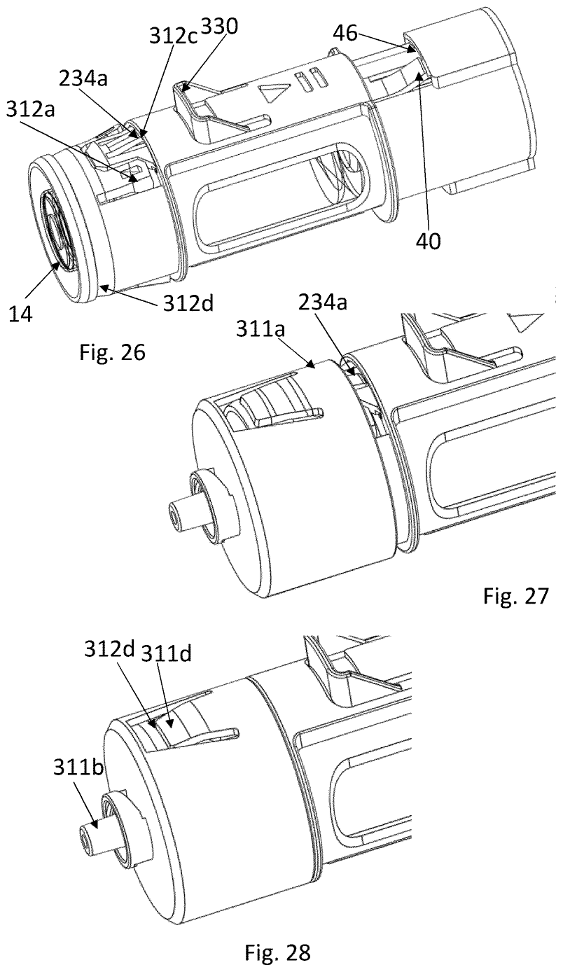

In one embodiment shown in the cross section of FIG. 20, the resilient member is a spring 144 arranged between the medicament container 14 and a distally facing surface 146 of the cassette body 12, 212. Since the medicament container 14 would not be connected directly to a needle, but instead connected to an infusion set the movement of the medicament container during the injection would not impact the injection experience for the patient and the movement could be masked by extending the cassette body 12, 212 over the medicament container 14. The spring may also reduce or even eliminate any rattling between the medicament container and the cassette body 12, 212.

FIGS. 21-28 and FIGS. 29-30 show another embodiment of the cassette 304; 403 without the slot 19, 219 of the cassette body as disclosed in the above examples. The rear cap 320 shares several features with the rear cap 20, such as the flexible arm 40, are describe above and will not be repeated here. Furthermore, the ridge 47 of the cassette body 312 disclosed here is also the same as the ridge 47 disclosed above. Instead of having two slider arms 24 as disclosed in the first embodiment, the rear cap 320 in this embodiment comprises one slider arm 234. The slider arm 234 comprises the actuator being the sliding button interface 330 for the user to slide the rear cap 320 in the distal direction. The sliding button interface 330 comprises a transversal protrusion so that a user can get a better grip on the sliding button interface 330. Alternatively, or additionally, the sliding button interface 430 comprises a gripping pattern, e.g., a rubber gripping pattern and/or ribs, as shown in FIG. 29. In one example, the slider arm 234, 434 is an integral part of the sliding button interface 330, 430. In one example, the slider arm 234, the sliding button interface 330, and the rear cap 320 are formed as a single component, as shown in FIG. 22. In this example, the slider arm 234, the sliding button interface 330, and the rear cap 320 are integral to one another. Alternatively, in another example, the slider arm 434 and the sliding button interface 430 are integral to one another, as shown in FIG. 30. In this example, the rear cap is not movable along with the slider arm 434. In this example, the rear cap is configured to prevent the medicament container from escaping from the distal end of the cassette body 412. For example, the rear cap is immovably attached to the distal end of the cassette body 412 and the slider arm 434 is configured to be moved in further distally relative to the rear cap. It should be noted that the movements of the rear cap as mentioned in the embodiments above, can be solely performed by the slider arm 434 instead in this example.

In this embodiment, the slider arm 234 comprises a proximal arm 234a extending in the direction of the longitudinal axis. The proximal arm 234a has a free end pointing towards the distal end of the cassette body 312. In this embodiment, the cassette body 312 comprises a proximally directed surface 312c. In a preferred example, the proximally directed surface 312c is the proximal edge of the cassette body 312, as shown in FIG. 22. The proximal arm 234a is configured to engage with the proximally directed surface 312c, as shown in FIG. 26, such that before the adapter 311 of the infusion set is operably attached to the medicament container 14, the slider arm 234 cannot move distally when the user accidentally moves the sliding button interface 330; 430 and the cassette body 312 is attached to the housing 3 of the medicament delivery device 1. It should be noted that most of the features of the infusion set 6 are identical in the above-mentioned embodiment, the main different features are arranged on the adapter 311; 411; thus, only the adapter 311; 411 will be explained in detailed here. The adapter 311; 411 comprises a body 311a, a connector 311d extending from a wall of the body 311a, and the protruding channel 311b, an inner channel coaxial with the protruding channel, as shown in FIGS. 23-24. The inner channel is configured to be at least partially positioned within the medicament container 14 when the adapter 311; 411 is operably attached to the medicament container 14. In this example, the inner channel comprises a shape distal end 311e, as shown in FIG. 24. The shape distal end 311e is configured to open a seal of the medicament container 14 when the adapter 311 is operably attached to the medicament container 14. The adapter 311; 411 is configured to be operably attached to the medicament container 14 via the connector 311d. In one example, the connector is configured to be attached to a neck of the medicament container directly. Alternatively, the connector 311d is configured to be attached to the cassette body 312. For example, the connector 311d is a snap-fit arm configured to be snap fitted to a groove 312d of the cassette body 312, as shown in FIG. 28 and FIG. 29. In one example, as shown in FIG. 29, the adapter 411 comprises gripping feature 411a, e.g., an indentation part, such that a user can easily grip and attach the adapter to the cassette body. Similarly, the

[symbol]

As mentioned above, the proximal arm 234a is configured to engage with the proximally directed surface 312c, as shown in FIG. 26, such that before the adapter 311 of the infusion set is operably attached to the medicament container 14; once the adapter 311 is attached to the medicament container 14, the body 311a of the adapter 311 presses the proximal arm 234a radially inward, as shown in FIG. 27. As a result, the proximal arm 234a is disengaged from the proximally directed surface 312c of the cassette body 312. Thus, once the cassette body 312 is attached to the housing 3, the rear cap 320 can be moved distally.

Furthermore, in another embodiment, the cassette body 312 comprises a container holder 312a, 312b for blocking the medicament container 14 from moving in the proximal direction of the cassette body 312. In a preferred example, the container holder 312a, 312b is a flexible arm 312a extending from a wall of the cassette body 312; the flexible arm 312a comprises a radially extending ledge 312b, as shown in FIG. 25. In this example, the medicament container 14 can be inserted into the cassette body 312 from the proximal end of the cassette body 312. The ledge 312b is configured to engage with the neck of the medicament container 14 when the medicament container 14 is inserted into the cassette body 312.

A medicament delivery device (such as an autoinjector) may generally include various other components. For example, a sensor unit which may recognize medicament delivery events, such as the needle inserted into an attachment portion of e.g., a pad, injection started, and medicament delivery operation ends, a memory unit which is configured to store the recorded data during the medicament delivery operation, a connectivity unit configured to transmit the stored data to a smart device or the network directly, a processing unit configured to control the entire system and processes the data before transmitting it, and/or user interface units that are configured to provide feedback to the patient, such as status LEDs, haptic, and/or audio feedback.

When the medicament delivery device is placed into the attachment portion, the sensors inside of the pad are configured to recognize the event and give feedback to the patient via haptic/visual or audio elements.

When the medicament delivery finishes, the sensors are configured to recognize the event and give feedback to the patient again. Further, the collected data is stored in the memory unit and may be transmitted to the smart device/network via the connectivity unit after the medicament delivery event finishes.

The sensor can be one of or the combination of the following: a mechanical switch, a Hall-effect sensor, an accelerometer.

The mechanical switch, hall-effect sensor, or accelerometer can be used for detection of the insertion of the a needle into an injection port.

The accelerometer can be used for detecting medicament delivery events.

Possible wireless communication methods include Bluetooth and Cellular Networks.

Bluetooth connectivity requires a smart device to transmit the stored data to the network and it requires a pairing action between the pad and the smart device before being able to use the supporting pad in case of 2-way connection. But it's a cheaper alternative and it requires less space on PCB. A 1-way connection does not require pairing.

The cellular network does not require any pairing process, it can be used as a plug-n-play device, no prior setup is needed, but it's more expensive and it requires more space on PCB.

Depending on the requirements of the product any of those two technologies can be used.

Such processing units may comprise a logic circuit or control unit including a microprocessor, microcontroller, programmable digital signal processor or another programmable device. The processing circuitry may also, or instead, each include an application specific integrated circuit, a programmable gate array or programmable array logic, a programmable logic device, or a digital signal processor. Where the processing circuitry includes a programmable device such as the microprocessor, microcontroller or programmable digital signal processor mentioned above, the processor may further include computer executable code that controls operation of the programmable device.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, antibodies, proteins, fusion proteins, peptibodies, polypeptides, pegylated proteins, protein fragments, protein analogues, protein variants, protein precursors, and/or protein derivatives. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

The inventive concept has mainly been described above with reference to a few examples. However, as is readily appreciated by a person skilled in the art, other embodiments than the ones disclosed above are equally possible within the scope of the inventive concept, as defined by the appended claims.

Some other aspects of the invention are defined in the following clauses.
1. A medicament cassette configured to accommodate a medicament container, the medicament cassette is attachable to a medicament delivery device for delivery of the medicament to an injection site via an infusion set connected to the medicament cassette, the medicament cassette reaching between a distal end and a proximal end along a longitudinal axis, the medicament cassette comprising:
a cassette body adapted to receive the medicament container in a cylindrical inner space of the cassette main body, the cassette body having a through-hole at a proximal end through which an adapter of the infusion set is connected to the medicament container;
at least one slider arm extending in the proximal direction; and wherein the at least one slider arm is arranged inside the cassette body;
an actuator movable with respect to the cassette body, the actuator is mechanically connected with the slider arm such that a movement of the actuator cause a sliding distal motion of the slider arm with respect to the cassette body.
2. The medicament cassette according to clause 1, wherein the at least one slider arm is an integral part of the actuator.
3. The medicament cassette according to clause 1 or 2, wherein the medicament cassette comprises a rear cap comprising a rear end plate at its distal end configured to prevent the medicament container within the cassette body from moving out from the distal end of the cassette body.
4. The medicament cassette according to clause 3, wherein the rear cap having at least one slider arm extending in the proximal direction; and wherein the actuator is mechanically connected with the rear cap such that a movement of the actuator cause a sliding distal motion of the rear cap with respect to the cassette body.
5. The medicament cassette according to clause 3 or 4 when dependent on clause 3, wherein the cassette body comprises at least one slot (19), the at least one slot has a main extension parallel with the longitudinal axis of the medicament cassette; and wherein the rear cap (20, 220) comprises at least one radially outwards extending protrusion (26) positioned in the at least one slot of the cassette body.
6. The medicament cassette according to any one of preceding clauses when dependent on clause 3, wherein the at least one slider arm (24) is an integral part of the rear cap (20, 220) extending in the proximal direction from the rear end plate of the rear cap (20, 220).
7. The medicament cassette according to any one of the preceding clauses when dependent on clause 3, wherein the rear cap comprises at least one flexible arm (40), the at least one flexible arm extends inclined radially outwards for initially preventing distal motion of the rear cap with respect to the cassette body.
8. The medicament cassette according to clause 7, wherein the flexible arms flex radially inwards by interaction with a receiving interface (10) of the medicament delivery device, whereby the rear cap is released from the cassette body and is movable in the distal direction with respect to the cassette body.
9. The medicament cassette according to clause 8, the rear cap comprising the actuator being a sliding button interface (30, 330) for a user to slide the rear cap in the distal direction.
10. The medicament cassette according to any one of the preceding clauses when dependent on clause 3, the rear cap comprising a proximally facing surface (42) to abut against a distal surface of the medicament container to prevent the medicament container from escaping out from the distal end of the cassette body.
11. The medicament cassette according to any one of the preceding clauses when dependent on clause 3, the at least one slot comprises a first portion (19a) and a second portion (19b) and a narrowing portion (19c) between the first portion and a second portion.
12. The medicament cassette according to clause 11, wherein the radially outwards extending protrusions (26) of the rear cap is longitudinally lockable in each of the first portion and the second portion.
13. The medicament cassette according to any one of clauses 1-6, wherein the cassette body comprises a through-hole (58) to receive the actuator in the form of a rotatable button (64), the rotatable button comprising at least one radial protrusion (70a, b) that reach the at least one slider arm of the rear cap inside the cassette body.
14. The medicament cassette according to clause 13, wherein the at least one slider arm comprises a cam surface (54) that the radial protrusion abuts against when the rotatable button is rotated, the interaction between the radial protrusion of the rotatable button and the cam surface cause the distal motion of the rear cap.
15. The medicament cassette according to clause 13 or clause 14, wherein the cassette body comprises two grooves (60a b) on a sleeve (62) of the cassette body surrounding the through-hole, and the rotating button comprises a at least one protrusion that is releasably engageable in each of the two grooves in two different rotational orientations of the rotating button with respect to the through-hole.
16. The medicament cassette according to any one of clauses 13 to 15, wherein the slot is uniformly shaped.
17. The medicament cassette according to any one of the preceding clauses, wherein the cassette body comprises radially inwards extending protrusions (57) configured to prevent the medicament container from escaping out from the distal end of the cassette body.
18. The medicament cassette according to any one of the preceding clauses, wherein the rear end plate comprises a longitudinal through-hole (38) for receiving a plunger rod from the medicament delivery device.
19. The medicament cassette according to any one of the preceding clauses, wherein the medicament cassette is attachable to a proximal end of the medicament delivery device by insertion of the medicament cassette in the proximal receiving interface (10) of the medicament delivery device, and a subsequent axial rotation of the medicament cassette (4, 204), whereby a proximally facing surface (46) of the cassette body engages with a distally facing surface (49) of the receiving interface (10) to longitudinally lock the medicament cassette in an attached position in the receiving interface (10).
20. The medicament cassette according to any one of the preceding clauses, the at least one slider arm (234) comprises a proximal arm (234a) extending in the direction of the longitudinal axis; wherein the proximal arm (234a) is engaged with the cassette body (312) before the infusion set is operably connected to the cassette body (312) such that the distal movement of the rear cap (320) relative to the cassette body (312) is prevented.

1-12. (canceled)

13. A medicament cassette configured to accommodate a medicament container, the medicament cassette being attachable to a medicament delivery device for delivery of the medicament to an injection site via an infusion set connected to the medicament cassette, the medicament cassette reaching between a distal end and a proximal end along a longitudinal axis, the medicament cassette comprising:
a cassette body adapted to receive the medicament container in a cylindrical inner space of the cassette main body, the cassette body having a through-hole at a proximal end through which an adapter of the infusion set is connected to the medicament container;
at least one slider arm extending in the proximal direction; and wherein the at least one slider arm is arranged inside the cassette body; and
an actuator movable with respect to the cassette body, the actuator is mechanically connected with the slider arm such that a movement of the actuator cause a sliding distal motion of the slider arm with respect to the cassette body.

14. The medicament cassette according to claim 13, wherein the at least one slider arm is an integral part of the actuator.

15. The medicament cassette according to claim 13, wherein the medicament cassette comprises a rear cap comprising a rear end plate at its distal end configured to prevent the medicament container within the cassette body from moving out from the distal end of the cassette body.

16. The medicament cassette according to claim 15, wherein the cassette body comprises at least one slot, the at least one slot having a main extension parallel with the longitudinal axis of the medicament cassette; and wherein the rear cap comprises at least one radially outwards extending protrusion positioned in the at least one slot of the cassette body.

17. The medicament cassette according to claim 15, wherein the at least one slider arm is an integral part of the rear cap extending in the proximal direction from the rear end plate of the rear cap.

18. The medicament cassette according to claim 15, wherein the rear cap comprises at least one flexible arm, the at least one flexible arm extends inclined radially outwards for initially preventing distal motion of the rear cap with respect to the cassette body.

19. The medicament cassette according to claim 18, wherein the flexible arms flex radially inwards by interaction with a receiving interface of the medicament delivery device, whereby the rear cap is released from the cassette body and is movable in the distal direction with respect to the cassette body.

20. The medicament cassette according to claim 19, wherein the rear cap comprising the actuator is a sliding button interface for a user to slide the rear cap in the distal direction.

21. The medicament cassette according to claim 13, wherein the at least one slot comprises a first portion and a second portion and a narrowing portion between the first portion and the second portion.

22. The medicament cassette according to claim 21, wherein the radially outwards extending protrusions of the rear cap is longitudinally lockable in each of the first portion and the second portion.

23. The medicament cassette according to claim 13, wherein the medicament cassette is attachable to a proximal end of the medicament delivery device by insertion of the medicament cassette in the proximal receiving interface of the medicament delivery device, and a subsequent axial rotation of the medicament cassette, whereby a proximally facing surface of the cassette body engages with a distally facing surface of the receiving interface, enables to longitudinally lock the medicament cassette in an attached position in the receiving interface.

24. The medicament cassette according to claim 13, wherein the at least one slider arm comprises a proximal arm extending in the direction of the longitudinal axis; and wherein the proximal arm is engaged with the cassette body before the infusion set is operably connected to the cassette body such that the distal movement of the rear cap relative to the cassette body is prevented.
